(19) United States
(12) Plant Patent Application Publication
(10) Pub. No.: US 2007/0113309 P1
(43) Pub. Date: May 17, 2007

(54) Apple tree named 'ZARI'

(75) Inventors: Johan Nicolai, Sint-Truiden (BE); Paul Van Laer, Heks (BE); Johan Keulemans, Korbeek-Lo (BE); Hendrik Eyssen, Holsbeek (BE); Els Pauwels, Amougies (BE); Inge De Wit, Lovenjoel (BE)

Correspondence Address: FOLEY AND LARDNER LLP;SUITE 500, 3000 K STREET NW, WASHINGTON, DC, 20007, US

(73) Assignee: BETTER3FRUIT N.V.

(21) Appl. No.: 11/529,551

(22) Filed: Sep. 29, 2006

(30) Foreign Application Priority Data
Nov. 16, 2005 (QZ) 2005/2293

(51) Int. Cl. A01H 5/00

(52) U.S. Cl. PLT/181

(57) ABSTRACT

A new and distinct apple tree named ‘ZARI’ particularly characterized by having early maturity of fruit (about 2 weeks earlier than ‘Gala’); good storability of fruit; good shelf life of fruit; and very crisp and juicy pulp of fruit.

### PRIORITY CLAIM

This application claims priority under 35 U.S.C. § 119(f) of the European Community Plant Variety Rights No. 2005/2293 filed Nov. 16, 2005.

### LATIN NAME OF THE GENUS AND SPECIES OF THE PLANT CLAIMED

Malus domestica

### VARIETY DENOMINATION

‘ZARI’

### BACKGROUND OF THE INVENTION

The present invention relates to a new and distinct variety of apple tree, botanically known as Malus domestica, of the Family Rosaceae, and hereinafter referred to by the variety name ‘ZARI’.

The new Malus variety is a product of a planned breeding program conducted by the inventors, Johan Nicolai, Paul Van Laer, Johan Keulemans, Hendrik Eyssen, Els Pauwels and Inge De Wit. The objective of the breeding program was to develop a new Malus variety which produced fruit with a crisp and juicy pulp, as well as, good storage capacity and shelf life.

The new Malus variety ‘ZARI’ originated from a cross made in a planned, controlled breeding program in Rillaar, Belgium in 1988. The female or seed parent is the Malus domestica variety ‘ELSTAR’ (patented, U.S. Plant Pat. No. 6,450 and Plant Breeder's Rights Nos.: BE 90002, DE APF 00007, FR 10412, GB 900030 and NL APP0060). The male or pollen parent is the Malus domestica variety ‘DELCORF’ (patented, Plant Breeder's Rights No. FR 112364).

The new seedling variety ‘ZARI’ was first fruited and selected for propagation and further testing by the inventors in 1994 in a controlled environment in Fernelmont (Franc-Waret), Belgium. Asexual reproduction of the new variety by grafting onto M9 rootstocks, was first performed in the winter of 1994-1995 in Gorsem, Belgium. Additional selection and asexual reproduction by the inventors occurred in 1999 and 2004 in Rillaar, Belgium. Asexual reproduction of ‘ZARI’ has demonstrated that the combination of characteristics as herein disclosed for the new variety are firmly fixed and retained through successive generations of asexual reproduction. The new variety reproduces true to type.

### BRIEF DESCRIPTION OF THE INVENTION

The following traits have been repeatedly observed and are determined to be unique characteristics of ‘ZARI’ which in combination distinguish this apple tree as a new and distinct variety:
1. early maturity of fruit (about 2 weeks earlier than ‘Gala’);
2. good storability of fruit;
3. good shelf life of fruit; and
4. very crisp and juicy pulp of fruit.

In comparison to the parental varieties, ‘ELSTAR’ and ‘DELCORF’, the new variety ‘ZARI’ differs primarily in the traits listed in Table 1.

TABLE 1

| | | Female Parent | |
|---|---|---|---|
| | New Variety | ‘ELSTAR’ | Male Parent |
| Characteristic | ‘ZARI’ | (PP6, 450) | ‘DELCORF’ |
| Fruit Shape | Oblong conical | Conic to slightly | Globose conical |
| | | oblong | |
| Fruit Skin | Yellow-green | Yellow-orange | Yellow |
| Color | | | |
| Overcolor of | Orange-red | Orange, red striped | Orange-red, |
| Fruit | | | striped |
| Flesh Color | Yellow | Creamy-white | Cream |
| | | tinged with yellow. | |

Of the commercial varieties known to the inventor, the most similar in comparison to ‘ZARI’ is the parental variety ‘DELCORF’ as compared in Table 1. In addition, the fruit of ‘ZARI’ is sweeter and has a better storability and shelf life than the fruit of ‘DELCORF’.

### BRIEF DESCRIPTION OF THE DRAWINGS

The accompanying photographs illustrate the overall appearance of the new apple tree ‘ZARI’ showing the colors as true as is reasonably possible with color reproductions of this type. Colors in the photographs may differ slightly from the color values cited in the detailed botanical description, which accurately describe the color of ‘ZARI’.

FIG. 1 shows a fruit bearing tree of ‘ZARI’ in the sixth growing season in August.

FIG. 2 shows a close-up view of the fruit, leaves and branches of ‘ZARI’ in the seventh growing season in August.

FIG. 3 shows a close-up view of mature fruit of ‘ZARI’.

FIG. 4 shows dissection views of mature fruit of ‘ZARI’.

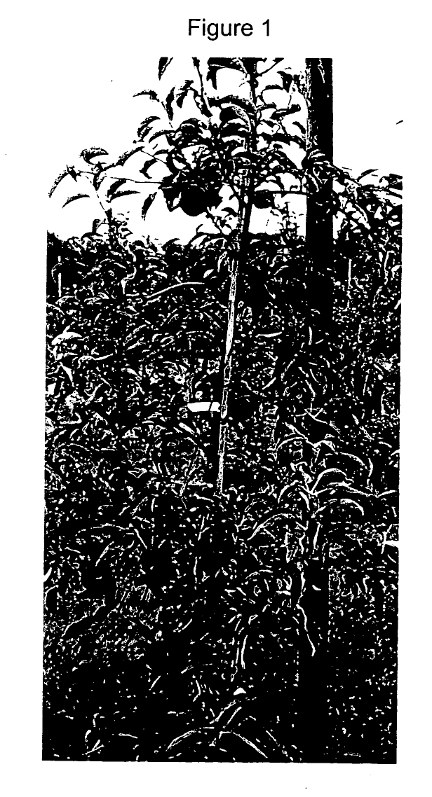

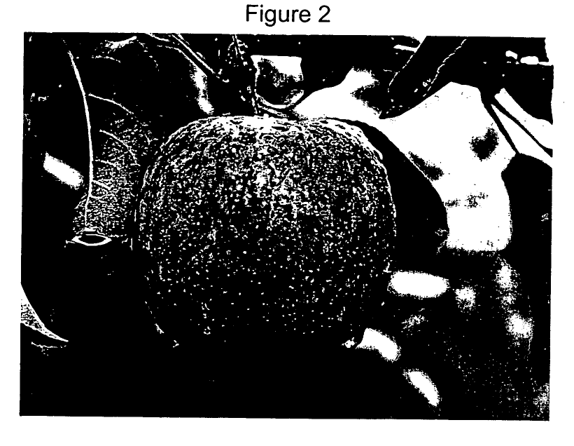

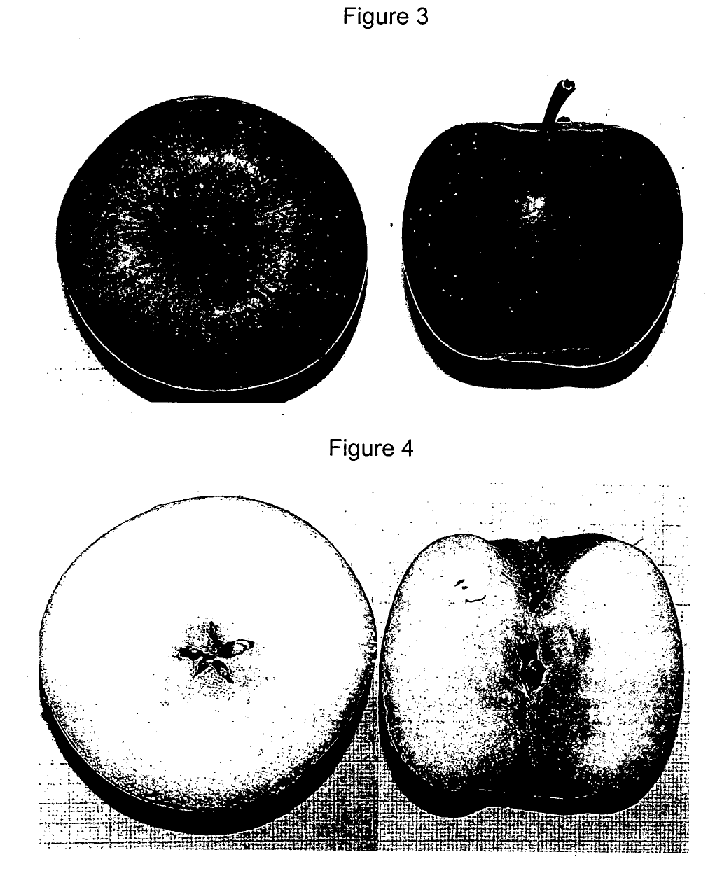

### DETAILED BOTANICAL DESCRIPTION

‘ZARI’ has not been observed under all possible environmental conditions. The phenotype of the new variety may vary with variations in environment such as temperature, light intensity, and day length without any change in the genotype of the plant.

The aforementioned photographs, together with the following observations, measurements and values describe trees of ‘ZARI’ as grown in the apple nursery in Rillaar, Belgium, under conditions which closely approximate those generally used in commercial practice.

Unless otherwise stated, the detailed botanical description includes observations, measurements and values based on five (5) year old ‘ZARI’ trees grown in the apple nursery in Rillaar, Belgium, from 2002 to 2006. Quantified measurements are expressed as an average of measurements taken from a number of trees of ‘ZARI’. The measurements of any individual tree, or any group of trees, of the new variety may vary from the stated average.

Color references are made to The Royal Horticultural Society Colour Chart (RHS; 4th edition, 2001), except where general colors of ordinary significance are used. Color values were taken under daylight conditions at approximately 3:00 p.m. in Rillaar, Belgium.
Classification:
   Botanical.—Malus domestica.
Parentage:
   Female or seed parent.—Malus domestica cultivar ‘ELSTAR’ (patented).
   Male parent.—Malus domestica cultivar ‘DELCORF’ (patented).
Propagation:
   Type.—Bench-grafting to M9 rootstocks.
   Locality where grown and observed.—Rillaar, Belgium.
Growing conditions:
   Light intensities.—Full sunlight.
   Temperature.—During day, grown in range of 2° C. to 23° C., and during evening, grow in range of −5° C. to 13° C. (based on the average month temperature).
Tree:
   Age.—Observed trees were five years old.
   Vigor.—Vigorous growth.
   Form.—Ramified and orthotropic spreading; branch angle is about 82°.
   Branching habit.—Main branches angle is 82.75°+/−7.34° with respect to trunk if allowed to grow naturally.
   Cropping behavior.—Average beginning production; good and constant production and flowering.
   Density.—About 2200 trees per hectare.
   Production.—7.7 kg per tree.
   Size at maturity.—Height: 254.00 mm+/−28.36 mm Spread: 174.00 mm+/−16.98 mm Trunk Diameter: 36.93 mm+/−3.71 mm.
   Surface texture.—Very rough.
   Bark color.—RHS 166A, greyed-orange.
   Trunk lenticels.—Length: 1.64 mm+/−0.15 mm Width: 3.23 mm+/−0.48 mm Color: RHS 158C, yellow-white Density: About 2 n °/cm².
Branches:
   Number per tree.—About 32.
   Length.—Varies due to shape of tree; up to a maximum of 101 cm+/−24.17 cm.
   Diameter (at 20 cm from the main stem).—14.48 mm+/−2.01 mm.
   Surface texture.—Rough.
   Color.—Mature (after about 3 years old): RHS 166A, greyed-orange New Growth: RHS 187A, greyed purple.
   Internode length.—41.53 mm+/−9.15 mm.
   Internode diameter.—11.8 mm+/−4.2 mm.
   Branch lenticels.—Length: 1.34 mm+/−0.23 mm Width: 2.25 mm+/−0.32 mm Shape: Oval Color: RHS 158C, yellow-white Density: About 2 n °/cm².
Leaves:
   Arrangement.—Alternate.
   Size.—Length: 11.28 mm+/−0.23 mm Width: 5.98 mm+/−0.32 mm.
   Overall shape.—Ovate, petiolated.
   Base shape.—Rounded.
   Apex shape.—Acute.
   Margin.—Serrate.
   Pubescence.—Upper surface None. Lower Surface: Slight.
   Attitude in relation to shoot.—Outwards.
   Color.—Mature leaves, upper surface: RHS 136B, green Mature leaves, lower surface: RHS 137D, green Immature leaves: RHS 144A, yellow-green (upper and lower surface).
   Venation.—Type: Pinnate Color: RHS 144B, yellow-green.
   Petiole.—Length: 3.05 mm+/−0.33 mm Diameter: 2.16 mm+/−0.15 mm Color: RHS 178A, greyed-red.
   Stipule.—Arrangement: free standing Length (distance of stipules from basal attachment of petiole): 7.12 mm+/−1.81 mm Width: 1.57 mm+/−0.55 mm.
   Spur.—Present: Yes Distance between each spur: 38.35 mm+/−7.65 mm Number of fruit per spur: 1.
Inflorescence:
   Time of beginning of flowering.—Medium (similar to ‘Golden Delicious’).
   Blooming period.—Short to medium.
   Fragrance.—Slight.
   Number of flowers per inflorescence.—5.10+/−0.31.
   Buds.—Arrangement: Alternate Length: 11.78 mm+/−0.98 mm Width: 5.17 mm+/−0.37 mm Color: RHS 187A, greyed-red.
   Petals.—Number per flower: 5 Length: 21.93 mm+/−2.59 mm Width: 14.89 mm+/−1.65 mm Overall shape: Obovate Apex shape: Rounded Base shape: Cunate Texture: Smooth (both surfaces) Margin: Entire Color: Open Flower Stage: Upper surface: RHS 70C, red-purple Lower surface: RHS N66C, red-purple Balloon stage: RHS N57D, red-purple.
   Sepals.—Number per flower: 5 Length: 9.84 mm+/−1.87 mm Width: 2.73 mm+/−0.47 mm Overall Shape: Triangular Apex Shape: Acute Base Shape: Truncate Texture: Slightly rough (both surfaces) Margin: Entire Color: RHS 143C, green (both surfaces).
Fruit:
   Keeping quality.—Very good for an early variety. The fruit keeps well on the tree. It can be stored in cold temperature conditions for up to 6 weeks without loosing firmness and juiciness. It has a shelf life up to 2 weeks without loosing firmness and juiciness.
   Maturity when described.—After 3 months in storage.
   Maturity period after full bloom.—About 4 months after full bloom.
   Date of picking.—Very early (end of August) (about 2 weeks earlier than ‘Gala’).
   Shape.—Oblong conical.
   Size.—Height: 54.71 mm+/−2.54 mm Width: 62.96 mm+/−1.82 mm Weight: 113.20 g+/−4.66 g.
   Stem.—Length: 36.23 mm+/−3.82 mm Width: 1.75 mm+/−0.12 mm Color: RHS 178A, greyed-red.
   Basin.—Depth: 0.90 mm+/−0.14 mm Width: 2.59 mm+/−0.23 mm.
   Calyx.—Depth: 1.38 mm+/−0.20 mm Width: 2.87 mm+/−0.22 mm.
   Crowning at calyx end.—Medium.
   Calyx tube.—Open.
   Skin.—Thickness: 0.21 mm+/−0.02 mm Texture: Slightly rough Overcolor of the skin: RHS N34A, orange-red Percentage of skin surface with overcolor: About 75% Pattern of overcolor: Striped Background color of the skin: RHS 154A, yellow-green.
   Lenticels.—Length: About 1.66+/−0.35 mm Width: About 0.96+/−0.21 mm Color: RHS 152A, yellow-green Density: 4.01+/−1.18 n °/cm².
   Flesh.—Color: RHS 11C, yellow Texture: Crisp and juicy Aroma: Strong Eating quality: Very good Firmness (at harvest, eat ripe): 7.28+/−0.65 kg/cm² Sugar content (at harvest, eat ripe): 15.43+/−1.06 Brix Acidity (at harvest, eat ripe): 9.30 g/l Malic acid.
   Core.—Symmetry of core: conical Distinctness of core lines: weak.
   Locules.—Number (per fruit): 5 Length: 15.47 mm+/−1.67 mm Width: 8.24 mm+/−1.08 mm Form: oval.
   Seeds.—Number per fruit: 4.80+/−1.79 Shape: Ellipsoid, elongated and pointed Length: 8.99 mm+/−0.47 mm Width: 4.58 mm+/−0.28 mm Color: RHS 200C, brown.
Reproductive organs:
Androecium:
   Stamen.—Number per flower: 17.20+/−2.86.
   Filament.—Length: 5.70 mm+/−1.40 mm.
   Anther.—Length: 2.46 mm+/−0.19 mm Diameter: 1.67 mm+/−0.19 mm Color: RHS 11C, yellow.
   Pollen.—Amount: Moderate Color: RHS 10A, yellow.
   Pollinators.—‘Gala’, ‘Golden Gem’, ‘Hillieri’.
Gynoecium:
   Pistils.—Number per flower: 5 Length: 10.37 mm+/−0.85 mm.
   Stigma.—Shape: Bulbous on top Color: RHS 151B, yellow-green.
   Style.—Color: RHS 151B, yellow-green.
Use: Fresh market.
Disease/pest resistance: No known resistance.
Winter hardiness: Tolerant to temperatures down to min. −10° C. without observed damage to wood and buds of dormant apple trees.
Drought/heat tolerance: Tolerant to temperatures up to min 40° C., growth is limited by drought periods without irrigation.

We claim:

1. A new and distinct apple tree plant named ‘ZARI’, substantially as illustrated and described herein.
